(19) United States
(12) Patent Application Publication
(10) Pub. No.: US 2010/0228118 A1
(43) Pub. Date: Sep. 9, 2010

(54) METHOD FOR IMAGE SUPPORT DURING THE NAVIGATION OF A MEDICAL INSTRUMENT AND AN APPARATUS FOR CARRYING OUT A MINIMALLY-INVASIVE INTERVENTION FOR THERAPY OF A TUMOR

(76) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/716,489

(22) Filed: Mar. 3, 2010

(30) Foreign Application Priority Data
Mar. 4, 2009 (DE) 10 2009 011 725.3

(51) Int. Cl. A61B 5/05; G06K 9/00; G01N 23/04

(52) U.S. Cl. 600/424; 382/132; 378/62

(57) ABSTRACT

A method for image support in the navigation of a medical instrument for a minimally-invasive intervention for therapy of a tumor in human body is proposed. A first current three-dimensional image dataset is recorded shortly before the intervention with an x-ray device showing the tumor. A second current three-dimensional image dataset is recorded with the x-ray device highlighting the blood vessels in the vicinity of the tumor while providing a contrast medium. The first and the second current image dataset are merged into a first merged image dataset. Two-dimensional images showing the medical instrument are repeatedly imaged with the x-ray device and/or an ultrasound device and/or determination of position data reflecting the three-dimensional position of the instrument by a position determination device registered with the x-ray device. The first merged image dataset is merged with the two-dimensional images and/or the position data to form a second merged image dataset.

### CROSS REFERENCE TO RELATED APPLICATIONS

This application claims priority of German application No. 10 2009 011 725.3 filed Mar. 4, 2010, which is incorporated by reference herein in its entirety.

### FIELD OF THE INVENTION

The invention relates to a method for image support in the navigation of a medical instrument for a minimally-invasive intervention for treatment of a tumor in the human body, especially an embolisation and/or ablation, with an x-ray device, especially including a C-arm, being used. In addition the method relates for an associated apparatus for carrying out a minimally invasive intervention for therapy of a tumor.

### BACKGROUND OF THE INVENTION

Cancers, in which at least one tumor can occur at different places in the human body, are among the most frequent causes of death. It is known in such cases that tumors and metastases can be treated by operative removal of the tissue affected. As a rule a chemotherapy or radiation therapy are also carried out here. However this involves complex processes which cause additional complications and pain for the patient without improving their quality of life and under some circumstances without being able to extend their life expectancy. In addition tumors exist which are not operable.

Methods have thus been proposed for treatment of tumors which manage with minimally-invasive intervention. In such a minimally-invasive method a medical instrument, especially a catheter, is introduced via a small opening in the body, guided to the tumor and vessels supplying blood to the tumor tissue and/or the tumor are obliterated or destroyed. In such cases there are essentially four known methods for minimally-invasive therapy of tumors.

In chemoembolisation blood vessels are explicitly closed up by introducing liquids which harden, small spirals or plastic particles (embolisate). The purpose of an embolisation is to isolate a tumor from the blood supply in order to “starve” it so to speak, which prevents its further growth and causes it to shrink or even destroys it. Chemoembolisation can be carried out before a planned operation for example, in order to facilitate the operation or to reduce blood loss during the operation. In specific cases the embolisation can however also be employed as the only therapy measure. Chemoembolisation is employed if the tumor is too large for an ablation or is in a location in which it cannot be treated by ablation. A combination of chemoembolisation and other methods of treatment is also possible. The embolisation is carried out percutaneously (through the skin) with there basically being two known variants.

In transarterial percutaneous catheter embolisation a nearby blood vessel, mostly the Arteria femoralis, is punctured under local anesthetic with a hollow needle (cannula). A catheter can then be introduced under x-ray control and pushed forwards into the artery that is to be closed up. Small particles or liquid substances are introduced through the catheter which block the blood flow.

With direct puncturing the tumor or the blood vessel to be blocked is punctured with the cannula directly through the skin, and the embolisate is introduced directly.

The diseases most frequently treated with embolisation are womb myomas, liver tumors or metastases, vertebral body tumors or metastases and glomus tumors. The case of a liver embolisation will be briefly discussed by way of example. Healthy liver tissue is supplied with 75% of its blood via the venose vessel system of the hepatic portal vein and with only 25% via the arterial blood stream of the gastric vein. By contrast liver tumors are predominantly supplied via the liver arteries. The chemo-embolisation method results in a closing off of the liver arteries, which causes the tumorous tissue to die. Normal liver tissue on the other hand is protected by sufficient portal venous blood flow. It is normal to introduce additional chemotherapeutics into the liver arteries. This means that the medicines which restrict the cell growth of the tumor are present in the liver tissue in a concentration up to 100 times higher than is the case with a systematic intravenous chemotherapy. The side-effects are also less marked. By suppressing the arterial blood flow the period over which the chemotherapeutics are active is also extended by between hours and weeks.

Typically in such a case chemoembolisation is carried out between two and three times at intervals of four to six weeks. In this case a marked reduction in the size of the tumor can occur so that the patient can subsequently undergo a local ablative procedure or an operation.

A second variant of minimally-invasive treatment of a tumor is themoablation, by means of high-frequency, microwave, ultrasound and/or laser energy for example. In this treatment the tumor cells are killed by high temperatures while healthy tissue remains protected. In Radio Frequency Ablation (RFA) an interventional radiologist introduces a thin needle into the patient's tumor with the aid of imaging technology. Radio frequency energy is transmitted from the point of the needle to the target tissue, where it generates great heat and thus kills the tumor. The dead tissue shrinks and slowly forms a scar. Depending on the size of the tumor, RFA can make it shrink or kill it, which prolongs the patient's life and significantly improves the quality of life. Since RFA involves a local method that does not harm healthy tissue the treatment can be repeated frequently.

The pain caused by tumors as well as other weakening symptoms are mitigated by the size of the tumor being reduced or by treatment of any new tumors arising. Although the tumors often do not cause any pain themselves, they can press on nerves or other important organs which sometimes causes great pain. Thermoablation can be used for small to medium-sized tumors, cf. also in this context the article by Zhengium. Liu, “Radiofrequency Tumor Ablation”, AJR: 184, April 2005, 1347-1352.

As well as radio frequency, other types of energy or supply of heat are of course also conceivable.

The method of cryoablation has a similar affect to that of thermoablation, since here too energy is conducted directly into the tumor through a medical instrument. Instead of killing off the tumor cells with heat however, the tumor is killed in cryoablation with the aid of an extremely cold gas. Cryoablation has been employed for many years by urologists, with medical instruments, especially needles, having become known in recent years that are so thin that an interventional radiologist can introduce them through a tiny incision in the body which means that open intervention is not required. A sort of “ice ball” forms around the needles which expands and kills the tumor cells. The reader is also referred here to the article by M. Beland, “Percutaneous Cryoablation of Symptomatic Extraabdominal Metastatic Disease”, AJR: 184, March 2005, 926-930.

Finally there is also radioembolisation, frequently also referred to as Selective Internal Radiotherapy (SIRT). It is very similar to chemoembolisation, with the vessels being closed off in the case of radioembolisation with radioactive micro balls. These little balls have roughly the diameter of five red blood cells and nest in the blood vessels of the tumor from where they give off their radiation, killing the tumor cells.

The radioisotope Yttrium-90 is frequently used in such cases. The radioactive particles are produced and brought to the clinic directly before the intervention. Yttrium-90 is especially suitable as a pure Beta radiator since the undesired radiation stress is small for living persons. 90% of the energy of the particle radiation is deposited in the tissue within a radius of appr. 9-11 mm. The comparative short half life requires only a short stay in a nuclear-medical clinic, for example 48 hours.

Radioembolisation is carried out as an image-controlled therapy by an interventional radiologist in conjunction with a specialist in nuclear medicine. After a local anesthetic has, been applied a thin catheter is introduced into the artery via a small incision in the groin. The catheter is advanced under fluoroscopy control into the liver artery for example. The radioactive isotope is injected through this catheter in the form of micro balls directly into those branches of the artery that supply the liver tumor. These micro balls remain stuck in the tumor vessels from where they emit radiation that kills the tumor cells. Since the radiation is restricted to this area, it can also be dosed correspondingly higher without presenting any significant risk to healthy tissue.

Radioembolisation involves a palliative treatment method which generally does not cure the cancer. Despite this patients benefit greatly from this treatment since their life expectancy is increased and thereby their quality of life improved by it. It involves a relatively new approach to therapy which has however already been shown to be successful in the treatment of primary tumors or metastases. With this method of treatment fewer side effects have been identified than with conventional chemotherapies for example. The main side effect is tiredness, which can last for between seven and ten days.

Radioembolisation is used for example for hepatocellular carcinomas, for cholangiocellular carcinomas and for liver metastases, such as of intestinal and breast cancer or other malignant tumors for example. The liver may not have been irradiated percutaneously beforehand, it must have an intact function and not have too great an ateriovenous shunt volume.

With regard to radioembolisation, the reader is also referred to the article by Bruno Sangro, “Radioembolisation—A New Treatment for Primary and Secondary Liver Tumors”, European Oncological Disease 2006, Page 36-38.

The known general procedure if cancer is suspected is currently initially a series of tests which can contribute to diagnosis, for example blood tests, physical examinations and a wide variety of imaging methods, especially taking x-rays, mammography, computed tomography, magnetic resonance and ultrasound. However a final diagnosis is mostly made after a biopsy.

In a biopsy a tissue sample is taken from the tumor or other abnormality and then investigated by a pathologist. They can establish the type of cancer involved and the speed at which this is likely to spread. Needle biopsies are mostly carried out with the aid of an x-ray method (fluoroscopy), computed tomography, ultrasound or magnetic resonance.

As soon as the type of tumor or metastasis involved is established, a decision about a therapy is taken. In the case of minimally-invasive therapy the doctor conducting the treatment will generally determine the tumor volume, based on three-dimensional CT or MR images, and thereby plan the therapy.

Subsequently the medical instrument is guided with the aid of two-dimensional x-ray images, i.e. typically the needle for percutaneous intervention or the catheter for the transarterial procedure.

Imaging support in the navigation of a medical instrument for such a minimally-invasive intervention is undertaken here, as mentioned, mostly by means of two-dimensional x-ray images (fluoroscopy), since these can be recorded in real time. However these are mostly of lower quality, so that the tumor itself and especially also the surrounding blood vessels which are not only of importance for a method using a catheter, can only be recognized with difficulty or not at all and the navigation of the medical instrument turns out to be complicated.

### SUMMARY OF THE INVENTION

The underlying object of the invention is therefore to specify a method with which a more precise and improved guidance of a medical instrument is possible in a minimally-invasive intervention at a tumor, especially an ablation or an embolisation.

To achieve this object, in a method of the type stated above, there is provision for the following inventive steps:
Shortly before the intervention, recording a first current, especially three-dimensional, image dataset in an imaging technology which clearly shows the tumor with the x-ray device,
Recording a second current, especially three-dimensional, image dataset in an imaging technology which highlights the blood vessels in the vicinity of the tumor, especially while providing a contrast medium, with the x-ray device,
Merging of the first and the second current image dataset into a first merged image dataset,
During the intervention repeated imaging of two-dimensional images showing the medical instrument with the x-ray device and/or an ultrasound device and/or determination of position data reflecting the three-dimensional position of the instrument by means of a position determination device registered with the x-ray device,
Merging of the first merged image dataset with the two-dimensional images and/or the position data to form a second merged image dataset,
Display of the second merged image dataset.

It is thus proposed, by a skillful merging of three different images, to supply the person carrying out the intervention with all the relevant information at a sufficient level of accuracy. Ultimately a display is created with the inventive method which makes possible a clear representation of the size of the tumor and/or activity (first image dataset), of the vessel structure (second image dataset) and of the position of the medical instrument. In this way a fast, secure and qualitatively-better minimally-invasive treatment of tumors and metastases results.

Shortly before the intervention, especially immediately before the intervention, the first and the second current image dataset are recorded and merged together. In this context the term shortly is to be understood as when the patient is already in the position intended for the intervention on an appropriate patient bed, so that the current image datasets can actually reproduce the situation to be presented to the person carrying out the intervention—by contrast with data that might already be out of date. The first image dataset in this situation is recorded in a recording technology clearly showing the tumor. Such recording technologies which allow the tumor to be identified and its size and/or activity to be represented are well known. The so-called soft tissue images might be given as an example. To record an image of the second current image dataset a recording technology highlighting the blood vessels in the vicinity of the tumor is used. In this case a high-contrast image in three dimensions with the introduction of contrast media is especially suitable. The two current image datasets, i.e. the three-dimensional soft-tissue image dataset and the three-dimensional high contrast image dataset for example are merged together, which is a simple operation because of the image is being recorded with the same x-ray device and thus the mandatory registration of the two image datasets. It should be pointed out even at this juncture that if a movement of the patient has occurred for example, other merging methods known in the prior art can be employed, i.e. registration on the basis of landmarks, segmentation or the like for example. However algorithms for movement correction can also be used especially advantageously, a practice which also applies especially to the second merging (with the two-dimensional images and/or the position data). Methods for movement correction are generally known in the prior art and do not need to be presented in greater detail here, nor do the various known merging methods.

Thus the first merged image dataset, which clearly shows both the relevant blood vessels and also the tumor, is available directly before the intervention. In this case there can expediently be provision for the first merged image dataset to already be displayed for planning the intervention and thus be able especially advantageously to supply current information for timely planning of the intervention.

The following steps of the method—recording of two-dimensional images that show the instrument or determination of position data, merging of the first merged image dataset with the two-dimensional images and/or the position data and display of the second merged image dataset are carried out repeatedly during the intervention, in particular essentially continuously, so that it is possible to follow the medical instrument in real-time. In this case three variants are essentially specified as to how information to be merged with the first merged image dataset about the position of the medical instrument can be obtained.

Initially it is possible to record a two-dimensional x-ray image with the x-ray device (fluoroscopy). In this case the medical instrument can especially be provided with x-ray markers if it were otherwise not able to be detected clearly enough. The procedure has the advantage that the images are recorded anew with the same x-ray device so that once again registration is already available and merging is possible in an especially simple manner. However it is also conceivable to record the two-dimensional images using an ultrasound device, i.e. an extracorporeal or intracorporeal ultrasound device for example, with or without ultrasound contrast media. It is especially advantageous in such cases for the ultrasound device to be already registered with the x-ray device so that the merging can also be undertaken in a simple manner. It is naturally also conceivable to use other ways of registering the two-dimensional images of the ultrasound device with the first merged image dataset, for example segmentation methods, the use of anatomical landmarks and suchlike.

In addition or as an alternative there can be provision for position data of the instrument to be recorded by means of a position determination device registered with the x-ray device. Three-dimensional position data of the instrument will thus be determined by the position determination device. Since the position determination device is registered with the x-ray device, it is a simple matter to display the medical instrument—for example as an icon—to create the second merged image dataset in the first merged image dataset. In particular in such cases the position data, if it is stored over time, can be viewed as a four-dimensional data set so that in an expedient further embodiment of the inventive method it is also possible to determine the movement path of the medical instrument and represent it in the second merged image dataset. The intervention can thus be followed at all times. It should be pointed out that the position can naturally also include the orientation of the instrument.

With such a position determination system there can be provision for at least one sensor belonging to the position determination system to be arranged on the medical instrument, with the aid of which the position of the instrument can be determined. In order to record patient movements and make the appropriate movement corrections, there can be provision for the position determination device to include a reference sensor for detecting patient movements, with the measurement data of the reference sensor being taken into account during the determination of the second merged image dataset. In this way it is still possible, despite the position data in the final analysis only including one position of the medical instrument, to still record movements of the patient and make a corresponding correction possible.

Frequently-used position determination devices that are known in the prior art are electromagnetic position determination devices which can also be used within the framework of current invention.

In such cases there can be provision particularly advantageously for a position determination device to be used that is integrated into the x-ray device. Such an embodiment is described for example in the German patent application published at a later date with the file reference DE 10 2008 032 313.6, with this publication hereby falling fully within the scope of the content disclosed by the present invention. The medical apparatus described in said invention which is especially suitable for minimally-invasive interventions, includes a patient support table, an imaging medical device (especially an x-ray device) as well as a localization and navigation device integrated therein. This is especially advantageous to the extent that, as already mentioned, the registration between the x-ray device and the position determination device is then a mandatory component of the overall apparatus in the final analysis.

A further advantage of the use of a position determination device is also that the radiation strain on the patient can be reduced, since fewer or even no two-dimensional x-ray images at all are required any longer.

A workflow for minimally-invasive treatment of a tumor using the inventive method for image support can for example appear as follows. Initially three-dimensional x-ray images are taken to obtain the first image dataset which shows the tumor clearly enough for the tumor tissue to be able to be identified. Thereafter the tumor boundaries can be defined in a representation of the first image dataset or where necessary also automatically by a computing device. Then the imaging of the second current three-dimensional image dataset is carried out with the x-ray device, with in this case the blood vessels that supply the tumor being highlighted. Such methods can especially involve a high-contrast image with the addition of a contrast medium. The first current image dataset is then merged with the second current image dataset and the first image dataset obtained can be displayed in order to again carry out planning of the minimally-invasive intervention on this.

During the intervention the navigation of the medical instrument for tumor treatment is now undertaken, with two-dimensional images and/or position data of the position determination system being recorded for imaging support and with the first merged image dataset merged into a second merged image dataset which will then be displayed. For example in the embolisation the medical instrument, especially the catheter, can be guided under constant visual control into the blood vessels to be obliterated. Once the destination is reached, the treatment is then undertaken, for example the introduction of the embolisate or the supply of the ablation energy, again under the control of the second merged image dataset reflecting the current position of the medical instrument.

Thereafter it is possible to check the success of the treatment once again with a three-dimensional image with the x-ray device, whereby once again especially three-dimensional image datasets which clearly show the tumor (soft tissue image) and/or three-dimensional image datasets which highlight the blood vessels (high contrast image) are created.

After the end of the minimally-invasive treatment it is usual after around three to five days to prepare further control images, for example by means of computed tomography, magnetic resonance, PET, SPECT, ultrasound, optical coherence tomography, PET computed tomography, SPECT computed tomography, PET magnetic resonance or SPECT magnetic resonance. If the treatment was successful the patient can be discharged from the clinic.

In a further embodiment of the inventive method there can be provision for an x-ray device with an integrated treatment apparatus, especially an ablation apparatus and/or an embolisation apparatus, to be used. Thus the actual treatment apparatus can be integrated into the x-ray device in order to further simplify control and handling.

In a further expedient embodiment of the inventive method there can be provision for an x-ray device with a C-arm arranged on an articulated-arm robot to be used. In this case the articulated-arm robot can have four degrees of freedom, especially six degrees of freedom. The use of an articulated-aim robot makes possible not only an especially flexible method of operation, but simultaneously improves access to the patient since an especially large number of positions of the C-aim, especially also a “park position” can be realized.

There can also be provision for the medical instrument to be guided on an articulated-arm robot, which especially has at least four degrees of freedom, preferably six degrees of freedom. An especially exact control of the medical instrument, i.e. of the catheter or the needle, is then also possible.

If the tumor is located in an area in which it is subjected in particular to the cyclic movements of the patient in a relevant manner, there can be provision for the image datasets and/or the two-dimensional images and/or the position data to be recorded with EKG or breathing triggering. Therefore images/data can also typically be created in this way which always correspond to the same breathing phases and/or heart phases and are thus especially easy to compare and to merge.

Frequently image datasets already recorded are available, especially from the time at which the tumor was diagnosed, which frequently show the tumor in high quality but which are not up-to-date however. The information of the image datasets recorded beforehand can inventively advantageously be used for image support during treatment if, during the merging of the first merged image dataset, a previously recorded, especially three-dimensional image dataset is also merged into the first image dataset. In particular there can be provision for the first current image dataset, which actually also shows the tumor clearly, to initially be registered or merged with the previously recorded image dataset in that an intermediate merged image dataset is created. This intermediate merged image dataset which actually also contains the data of the first image dataset is then merged with the second image dataset to form the first merged image dataset. The registration of the previously recorded image dataset with the first current image dataset can be undertaken using the normally known registration techniques, in particular however landmarks, especially of the tumor itself, can also be used. The previously recorded image dataset in such cases can for example have been created by means of computed tomography, magnetic resonance, PET, SPECT, ultrasound, optical coherence tomography, PET-CT, SPECT-CT, PET-MR or SPECT-MR. PET-CT images which have been created by means of a combined PET-computed tomography apparatus and which can show the tumor particularly clearly are preferred here.

As well as the method, the present invention also relates to an apparatus for carrying out a minimally-invasive intervention for the therapy of a tumor, comprising an x-ray device with a C-arm, a treatment apparatus, especially an ablation apparatus and/or and embolisation apparatus, as well as a control device, especially a central control device, which is embodied for carrying out the inventive method. With such an apparatus an especially advantageous image monitoring of a minimally-invasive intervention and an outstanding support in the navigation of the medical instrument is especially possible. In such an apparatus the patient is for example first positioned on a patient support table with a patient support plate, whereupon, especially immediately before the intervention, the first and the second image datasets are recorded by means of an x-ray device which is controlled in the appropriate manner by the control device. The merging of the two current image datasets into a first merged image dataset is now also undertaken in the control device. The medical instrument which is part of the treatment device, i.e. a catheter or a needle for example, is then introduced to carry out the intervention. During this intervention the x-ray device can be controlled by the control device, to record two-dimensional x-ray images which are then merged with the first merged image dataset to form a second merged image dataset so that image support is provided during the navigation of the medical instrument. Here too the merging can take place in the control device. As an alternative or in addition, for recording two-dimensional x-ray images during intervention, the inventive apparatus can also comprise a position determination device for determining position data of the medical instrument, with the device especially advantageously able to be integrated into the x-ray device as already described above. Also conceivable is an ultrasound device, especially one integrated into the x-ray device. The control device is embodied in these cases to use the position data to reproduce the position of the medical instrument in the second merged image dataset or to register and to merge two-dimensional ultrasound images with the first merged image dataset.

If the treatment apparatus is also especially advantageously integrated into the x-ray device, then ultimately the x-ray device itself also forms the inventive apparatus for carrying out a minimally-invasive intervention, with all components—the x-ray device, the treatment apparatus, the position determination device and/or the ultrasound device being matched to each other in the optimum way and in particular able to be controlled by the same control device. Registration is then provided between the different systems which makes merging and thereby image support easier.

In an advantageous further embodiment of the inventive apparatus there can be provision for the and/or the medical instrument to be guided on an articulated-arm robot with at least four, especially six degrees of freedom. The C-arm can thus be controlled especially flexibly and access to the patient is facilitated. The medical instrument too can be advantageously guided in an especially exact and flexible manner. In this case there can be provision for both the C-arm and also the medical instrument or the corresponding articulated-arm robot to be able to be controlled by the control device.

### BRIEF DESCRIPTION OF THE DRAWINGS

Further advantages and details of the present invention emerge from the exemplary embodiments presented below as well as with reference to the drawings. The figures show:

FIG. 1 a flowchart of the inventive method,

FIG. 2 a view of an inventive apparatus, and

FIG. 3 a basic sketch showing the most important components of the inventive apparatus.

### DETAILED DESCRIPTION OF THE INVENTION

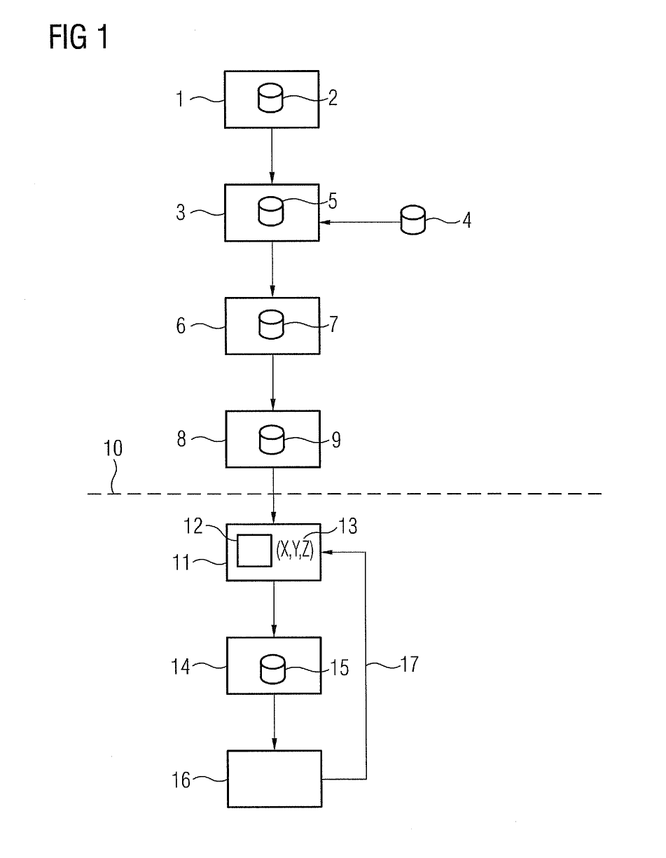

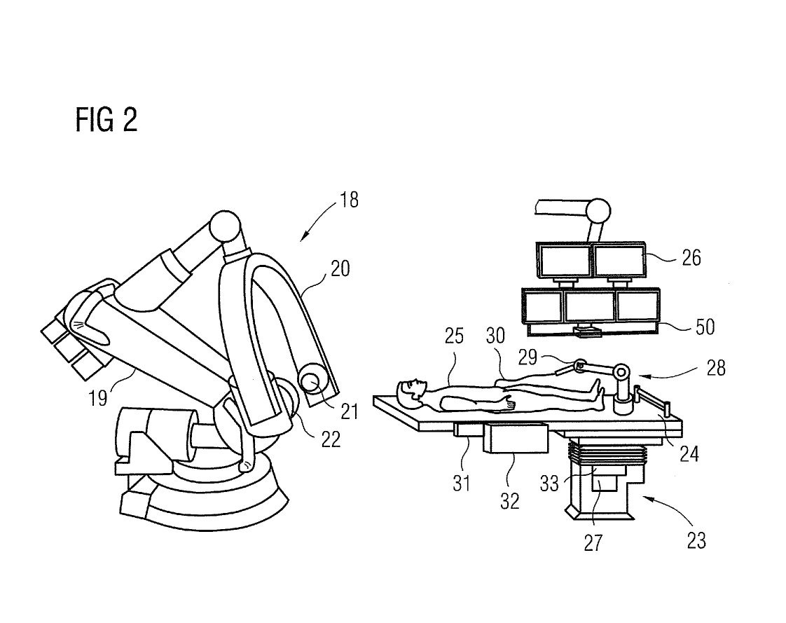

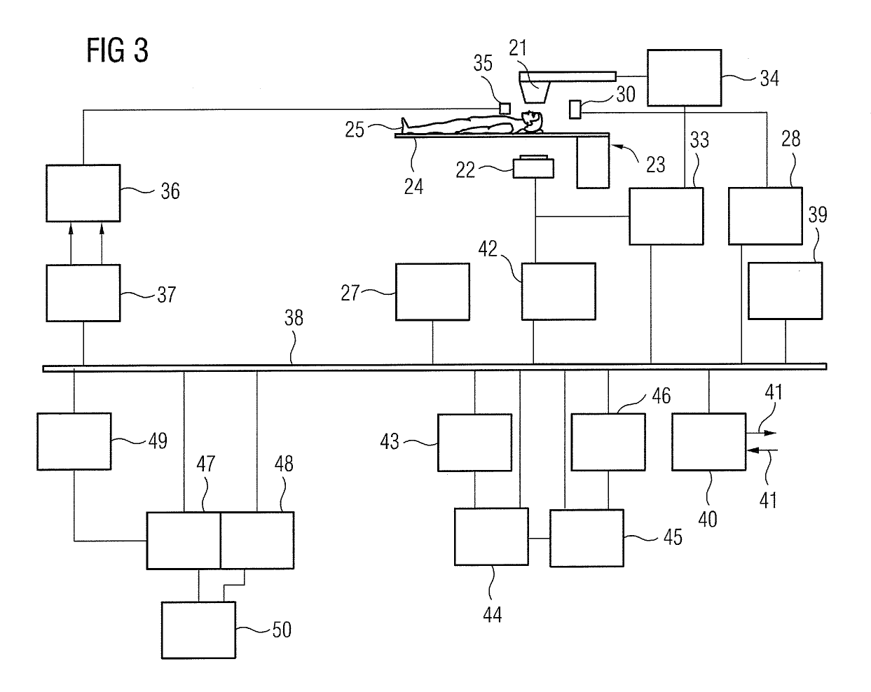

FIG. 1 shows the flowchart of the inventive method. It is used for image support in the navigation of the medical instruments during a minimally-invasive intervention for treating a tumor in the human body. In such cases ablation and embolisation should be mentioned in particular, as has been stated above. The method depicted in FIG. 1 is carried out on an x-ray device into which a treatment apparatus and an electromagnetic position determination device are integrated. This x-ray device on which the inventive method is carried out is described in greater detail below with reference to FIGS. 2 and 3.

At the beginning of the procedure the patient is initially positioned on a patient support table. The x-ray device comprises a C-arm attached to a floor-mounted articulated-arm robot with six degrees of freedom, with the aid of which it is possible to also make three-dimensional images. Naturally floor-mounted or wall-mounted C-arms are also conceivable.

Directly before the intervention is carried out the inventive method now begins with a step 1 in which a first current three-dimensional image dataset is recorded with the x-ray device in a recording technique clearly showing the tumor. In the present example a recorded image of soft tissue is involved here. If necessary there can be provision for this first image dataset 2 to already be displayed on a display device in order to determine the current tumor volume or suchlike for planning the minimally-invasive intervention.

In the inventive method there then follows an optional step 3 if a previously recorded image dataset 4 exists. Such a previously recorded image dataset 4 can for example be a PET-CT image dataset on which a tumor can be seen especially clearly. The first image dataset 2 and the previously recorded image dataset 4 can then be merged into an intermediate merged image dataset 5. Widely-known methods can be used for registering the image datasets 2 and 4, with a landmark-based approach with the tumor shown especially clearly in the previously recorded image dataset 4 proving to be useful.

It should also be noted at this point that step 3 can also be integrated into step 8 to be discussed below, in which three image datasets are then merged together with one another.

In a step 6 a second current three-dimensional image dataset 7 is then recorded—once again immediately before the minimally-invasive intervention is carried out. For this step a recording technology is used which especially highlights the blood vessels around the tumor with a high contrast image with the addition of a contrast means being produced here.

In step 8 the first merged image dataset 9 is then determined, which can be produced by an indirect merging of the first current image dataset 2 and the second current image dataset 7, however if an intermediate merged image dataset 5 has already been determined in step 3 taking into account previously recorded image datasets 4, it is produced by merging of the intermediate merged image dataset 5 and the second current image dataset 7. Since the first current image dataset 2 and the second current image dataset 7 were recorded with the same x-ray device, they are registered here in any event. In the first merged image dataset 9 both the tumor and also the blood vessels supplying it are thus very clearly to be seen. If necessary there can optionally be provision for the first merged image dataset 9 to be displayed on a display device of the x-ray device so that further planning can be undertaken thereon.

The minimally-invasive intervention begins after step 8, as shown by the dashed line 10. During this minimally-invasive intervention there should now be continuous monitoring of where the medical instrument is located. To this end data is determined in a step 11 which specifies the position of the medical instrument. Two variants are envisaged for this purpose in the exemplary embodiment shown, which can both of course also be used simultaneously. On the one hand it is possible by means of the x-ray device to record a two-dimensional image 12 on which the medical instrument where necessary provided with x-ray markers is to be seen. On the other hand position data of the instrument can be determined by means of the position determination device already mentioned above. Since the position determination device is registered with the x-ray device, especially is actually integrated into the latter, the position data can also be easily set in relation to the first merged image dataset 9.

Thus as a consequence in the inventive method in step 14 a second merged image dataset 15 is determined which can arise from the merging of the first merged image dataset 9 with the second two-dimensional image 12 (which is actually easily possible because of the use of the same x-ray device) and/or by using the position data 13 to merge the instantaneous position of the medical instrument with the first merged image dataset 9. The result is the second merged image dataset 15 which consequently not only shows the tumor and the blood vessels supplying the tumor, but also the instantaneous position of the instrument. It should be pointed out at this point that the position of the medical instrument—especially when a position determination device is used—can also be followed over time and thus the movement path of the instrument can be included in the second merged image dataset 15.

In a step 16 the second merged image dataset 15 is then displayed on a display device to the person carrying out the intervention. This person can thus orient himself outstandingly well in the navigation of the medical instrument since the position of the instrument relative to the tumor and to the blood vessels is visible to him at all times.

Steps 11, 14 and 16, as indicated by the arrows 17, are constantly repeated during the minimally-invasive intervention, so that the position of the medical management can be traced in relation to the tumor and to the blood vessels at least approximately in real-time and it is possible to keep the person carrying out the intervention outstandingly well informed.

It should also be pointed out at this juncture that movements of the patient during the method steps 1, 3, 6 and 8 or during the minimally-invasive intervention can also be taken into account by employing the appropriate movement correction algorithms. In particular there can be provision for the position determination device to include a reference sensor for detecting patient movements, with the measurement data of the reference sensor being taken into account during the determination of the second merged image dataset. Such a sensor can for example be arranged on the patient in the stomach area, to be able to record the breath movement of the patient.

In any event, if the tumor is in an environment adversely affected by cyclic movements of the patient, i.e. the breathing and/or the heartbeat for example, there can be provision in the inventive method for any recordings or determinations of position data to be made on the basis of an EKG and/or breath trigger in order to ensure that the image datasets/images/position data to be merged are recorded in the respective same phase in the cyclically-repeating movement.

FIG. 2 now shows a view of an inventive apparatus for carrying out a minimally-invasive intervention for therapy of a tumor. It comprises an x-ray device 18 with a C-arm 20 arranged on a floor-mounted articulated-arm robot 19 with six degrees of freedom, on which a radiation source 21 and a detector are provided opposite one another. The x-ray device 18 further comprises a patient support table 23 with a patient support plate 24 on which a patient 25 lies during the intervention.

Furthermore a display device 50 with a number of monitors 26 is provided.

Integrated into the x-ray device 18 and only shown schematically is a position determination device 27, in this example an electromagnetic position determination device. Also integrated into the x-ray device 18 is a treatment apparatus 28, i.e. an ablation apparatus and/or embolisation apparatus for example, which in the present example includes an articulated-arm robot 29 arranged on the patient support plate 24 with six degrees of freedom as well as the medical instrument 30 arranged thereon. Naturally, especially in the case in which the medical instrument 30 is a catheter, other embodiments are also conceivable.

Also provided at the side of the patient support plate 24 are operating consoles 31 and 32 for the x-ray device 18 and the treatment apparatus 28 close to the patient. All components of the inventive apparatus are controlled via a control device 33 likewise shown only schematically which is also embodied to carry out the inventive method, as has been described for example in relation to FIG. 1.

FIG. 3 finally shows in the form of a schematic diagram a system overview of important components of the inventive apparatus. The figure again shows the patient 25 on the patient support plate 24 of the patient support table 23, the radiation source 21 and the x-ray detector 22. The radiation source 21 is supplied with the necessary voltage via a high-voltage generator 34. Also shown is the treatment apparatus 28, with the medical instrument 30 and the electromagnetic position determination device 27 once more only shown schematically. Further indicated is a movement detector 35 not shown in FIG. 3, which in addition to the physiological sensors not shown in any great detail here, can also be used for example to determine the time for the EKG/breath gating. For this purpose the movement detector is connected via a corresponding interface 36 to the corresponding movement and gating processor 37.

All components shown here are controlled by the control device 33, with the communication between the different components/apparatuses occurring via a data bus 38. Connected to this bus is an image and data store 39 on which for example a previously recorded image dataset can be stored. In any event a DICOM interface 40 for patient data and image data is also provided which, as indicated by the arrow 41, also allows communication with other apparatuses, for example via a network.

In addition to the preprocessing unit 42 for x-ray images, a few further units/components/processors are provided which can be used when carrying out the inventive method and when carrying out the minimally-invasive intervention or its planning. Thus the x-ray images/image datasets recorded by the x-ray device 18 are further processed in an image processing unit 43 for x-ray images. A specific 3D processor 44 for soft tissue images can also be provided. Movement corrections can be undertaken for example in a calibration and image correction unit 45.

Of particular relevance for the present invention is the image merging and reconstruction unit 46. Not only the reconstruction of three-dimensional datasets is undertaken here but also the merging of a number of image datasets into merged image datasets, as has already been explained in greater detail with reference to FIG. 1. The display device 50 is finally controlled by a corresponding display unit 47 and via a user interface 48.

It should finally also be pointed out that the system presented here can also include an ontological therapy planning unit 49 via which the advance planning of the therapy and especially of the minimally-invasive intervention can be undertaken.

In this way an x-ray device can be realized which through integration of a diverse range of devices can offer an especially suitable tool for minimally-invasive therapy of tumors.

Naturally system architectures other than that shown here are also conceivable. In particular it should be remarked in conclusion that the inventive apparatus for carrying out a minimally-invasive intervention for therapy of a tumor can also include an ultrasound device if ultrasound images are also to the incorporated into the system.

1.-18. (canceled)

19. A method for an image support in a navigation of a medical instrument for a minimally-invasive intervention for treatment of a tumor in a patient, comprising:
recording a first current three-dimensional image dataset before the intervention with an x-ray device that shows the tumor;
recording a second current three-dimensional image dataset with the x-ray that highlights blood vessels in a vicinity of the tumor while providing a contrast medium;
merging the first and the second current image datasets into a first merged image dataset;
repeatedly imaging two-dimensional images showing the medical instrument during the intervention with the x-ray device and/or an ultrasound device;
merging the first merged image dataset with the two-dimensional images to form a second merged image dataset; and
displaying the second merged image dataset.

20. The method as claimed in claim 19, wherein a movement correction of the patient is performed in at least one of the first and the second merging steps.

21. The method as claimed in claim 19, wherein the x-ray device is integrated with a treatment device.

22. The method as claimed in claim 19, wherein the x-ray device comprises a C-arm arranged on an articulated-aim robot.

23. The method as claimed in claim 22, wherein the medical instrument is guided on the articulated-arm robot.

24. The method as claimed in claim 19, wherein the three-dimensional image datasets or the two-dimensional images are recorded with an EKG or a breath trigger.

25. The method as claimed in claim 19, wherein a movement path of the medical instrument is determined and presented in the second merged image dataset.

26. The method as claimed in claim 19, wherein the first merged image dataset is shown for planning the intervention.

27. The method as claimed in claim 19, wherein a previously recorded three-dimensional image dataset is merged with the first merged image dataset.

28. A method for an image support in a navigation of a medical instrument for a minimally-invasive intervention for treatment of a tumor in a human body, comprising:
recording a first current three-dimensional image dataset before the intervention with an x-ray device that shows the tumor;
recording a second current three-dimensional image dataset with the x-ray that highlights blood vessels in a vicinity of the tumor while providing a contrast medium;
merging the first and the second current image datasets into a first merged image dataset;
repeatedly determining a position data reflecting a three-dimensional position of the instrument by a position determination device registered with the x-ray device;
merging the first merged image dataset with the position data to form a second merged image dataset; and
displaying the second merged image dataset

29. The method as claimed in claim 28, wherein the position determination device is integrated into the x-ray device.

30. An apparatus for carrying out a minimally-invasive intervention for therapy of a tumor, comprising:
an x-ray device with a that:
records a first current three-dimensional image dataset before the intervention that shows the tumor;
records a second current three-dimensional image dataset that highlights blood vessels in a vicinity of the tumor while providing a contrast medium;
repeatedly images two-dimensional images showing the medical instrument during the intervention;
a treatment apparatus; and
a control device that:
merges the first and the second current image datasets into a first merged image dataset;
merges the first merged image dataset with the two-dimensional images to form a second merged image dataset.

31. The apparatus as claimed in claim 30, wherein the treatment apparatus is integrated into the x-ray device.

32. The apparatus as claimed in claim 30, further comprising a position determination device integrated into the x-ray device that determines a position data of the medical instrument.

33. The apparatus as claimed in claim 32, wherein the first merged image dataset is merged with the position data of the medical instrument to form the second merged image dataset.

34. The apparatus as claimed in claim 32, wherein the position determination device is an electromagnetic position determination device.

35. The apparatus as claimed in claim 32, wherein the position determination device comprises a reference sensor for detecting a patient movement in determining the second merged image dataset.

36. The apparatus as claimed in claim 30, further comprising an ultrasound device integrated into the x-ray device.

37. The apparatus as claimed in claim 30, wherein the C-arm or the medical instrument is guided on an articulated-arm robot with at least 4 degrees of freedom.
